(19) United States
(12) Patent Application Publication
(10) Pub. No.: US 2004/0209538 A1
(43) Pub. Date: Oct. 21, 2004

(54) Textile implant

(76) Inventors: Uwe Klinge, Montzen (BE); Bernd Klosterhalfen, Herzogenrath (DE); Boris Obolenski, Aachen (DE); Stefan Schneemelcher, Aachen (DE)

Correspondence Address: WILLIAM COLLARD, COLLARD & ROE, P.C., 1077 NORTHERN BOULEVARD, ROSLYN, NY, 11576, US

(21) Appl. No.: 10/481,948

(22) PCT Filed: Jun. 25, 2002
(86) PCT No.: PCT/DE02/02287

(30) Foreign Application Priority Data
Jun. 26, 2001 (DE) 101 30 289.4
Jul. 10, 2001 (DE) 101 32 762.5

(51) Int. Cl. A61F 2/02; B32B 5/02

(52) U.S. Cl. 442/59; 623/23.74; 606/151; 442/286; 442/396

(57) ABSTRACT

In surgery, mesh-shaped implants, so-called meshes, are used for supporting damaged body parts that are to be closed. These usually consist of multifilament polymers, more specifically of polyester and polypropylene. Post-surgery complications are frequent, though. The implant also becomes encapsulated as a foreign body within the tissue.

In order to enhance the tolerability of a used implant it is proposed that the implant comprises monofilament threads of polyvinylidene fluoride (PVDF) or of a PVDF derivative.

The invention relates to a textile implant material.

In medical engineering, more specifically in surgery, one is often confronted with the task of stabilizing damaged cavities or gaps in the human or animal body trunk using spacers and/or of durably closing damaged orifices.

In abdominal ventral or inguinal hernia surgery for example, it is often necessary to close the surgical incision in the trunk with a suture. A simple conventional suture however is subjected to high mechanical stresses, when coughing for example, and is at high risk as a result thereof. In such cases, a mesh-like implant (mesh) is therefore often inserted in the abdominal wall for mechanically supporting the suture. In the patient's body, these meshes are capable of taking forces in two or more directions, thus relieving the stress on the suture itself.

Currently, commercially available meshes mainly consist of multifilament polymers, more specifically of polyester and polypropylene having various structures.

Unfortunately, post-surgery complications often occur using implants of this type. In the course of time, the flexibility of the implant diminishes, the fabric hardens and an inflammatory foreign-body reaction in the human body may ensue, which results in aggressive scarring. This durably limits and/or painfully impairs the patient's mobility.

Moreover, the body tissue encapsulates the implant, which thus remains in a way a foreign body within the repaired structure. Although polypropylene and polyester are materials that are approved in surgery, they do not bond with the body. The body durably identifies these materials as foreign bodies and prevents them from homogeneously bonding with human tissue.

The inventor therefore addressed the problem of developing an implant that will not present the disadvantageous features indicated, or at least will present them to a lesser extent, thus promising to be better tolerated by the patient.

In accordance with the present invention, the resolution to this problem is achieved by a textile implant material that is comprised of monofilament threads of polyvinylidene fluoride (PVDF) or of a PVDF derivative.

Polyvinylidene fluoride (PVDF) refers to a class of materials exhibiting biological properties that are very advantageous for the application described herein. More specifically, as directly compared to polyester, it has a clearly improved hydrolysis resistance. Whereas the flexibility of polypropylene diminishes in the course of time, resulting in a hardened material, this phenomenon does not occur with PVDF so that, as a result thereof, the patient's mobility is not impaired as it is the case with polypropylene. PVDF is not known to be subjected to an ageing process.

Moreover, the textile properties of PVDF are stable within a temperature range of from −40° C. to +160° C. In the normal case, the implant within the patient's body will not be subjected to a temperature outside this range. Its friction resistance is similar to that of the polyamides and is thus considerably greater than that of the polyesters. Moreover, PVDF exhibits high resistance to many organic acids and mineral acids as well as to aliphatic and aromatic hydrocarbons, alcohols and halogenated solvents. Moreover, the inflammatory foreign-body reaction of the human tissue is clearly reduced as compared to polypropylene.

Moreover, in using monofilament threads, the total number of threads composing the implant may be reduced. This permits to advantageously reduce the total thread surface and, as a result thereof, the overall implant surface.

In an advantageous embodiment of the invention, the textile implant material is composed mainly or even completely of monofilament threads. As a result thereof, the advantageous properties of the monofilament structure come even more to the fore.

It is advantageous if the implant in accordance with the invention has a porous structure. As a result thereof, regrowing or ingrowing human tissue is allowed to spread in the pores, thus allowing not only for initially improved mechanical properties, but also for an enhanced overall integration of the implant in the given tissue structure. Encapsulation of the implant, which is often observed, is thus largely or even completely prevented.

It is particularly advantageous if the implant of the invention has a pore size of from 1 to 5 mm, preferably from 1 to 3 mm. It has been found out that, with a pore structure having these sizes, the advantageous mechanisms described occur to a particularly great extent.

In an advantageous embodiment, the implant in accordance with the invention is comprised of a woven fabric, a knit fabric, a knitting, a layered fabric, a braided fabric or a nonwoven fabric. These types of construction of the monofilament threads make certain that occurring forces be taken from a plurality of directions and be diverted toward a plurality of directions the best possible way within the implant of the invention. Furthermore, an implant of such design permits the surgeon to readily connect sutures thus giving him the greatest possible freedom to decide how to connect the implant to the neighboring tissue.

Particularly good results are obtained if the implant of the invention has a flexible structure. A plurality of advantages are to be mentioned here: on the one hand, a patient can sensorily feel a flexible implant of the invention to a reduced extent only as the body tissue surrounding the implant also behaves elastically, thus permitting to better ensure homogeneity of the elasticity in the region of concern. On the other hand, this also clearly reduces the risk of shearing or tearing the implant of the invention off the tissue it has been sutured to since tension peaks can be better diverted. Concurrently, this also reduces the probability that the patient will need further operation.

For selective applicability for various medical needs and/or objectives, it may also be advantageous if at least part of the implant of the invention comprises a resorbable material.

It is further advantageous if at least part of the implant comprises a biocompatible material. Independent of the mechanical compatibility of the implant used, another factor determining the successful integration of the implant in the body tissue is the biological compatibility. Here, the biocompatibility of the implant's surface is of paramount importance. Immediately upon placing the implant into the body trunk, a plurality of reactions occur between implant and tissue. The first reaction hereby is a physical one: biomolecules, proteins in particular, are uncontrolledly absorbed on the surface of the implant. These biomolecules, which are bound by absorption, appear to have changed their conformation in such a manner that they loose their biological activity or perform another type of unintended biological function. The conformation of the absorbed proteins now decisively influences cell adhesion and cell propagation on the surface of the implant. For example, individual protein molecules are intended to be converted to signal substances by intentional conformation changes or protein fragments acting as signal substances are released during catalytic (proteolytic) reactions. Accordingly, the biocompatibility of the implant depends to a considerable extent on the protein absorption being purposefully influenced.

In selectively inserting biocompatible, functional groups into the material, specific ligand-receptor interactions as they occur by their own between cells and extracellular matrix (ECM) are initiated. Using suited ligands for targeting the surface receptors of the endogenous cells makes the reaction of the organism to the implant controllable. This makes active integration possible. Ideally, even permanent human tissue integration may be achieved.

The implant may be configured so as to be provided, at least on its top and/or bottom side, with a net-like biocompatible material in one or several pieces. This permits to achieve strong support of the surrounding body tissue.

The implant in accordance with the invention also yields particularly good results if at least part of the implant comprises a coating. Since at least the initial reactions between implant and body tissue are mere surface reactions, the processes taking place here may be selectively influenced by providing the implant with a surface coating. Such type coatings are well known. Cell adhesion proteins may for example improve biocompatibility or other kinds of coatings such as a drug coating may reduce or even prevent infections and other post-operative complications.

For taking into consideration the elasticity of the surrounding tissue and, as a result thereof, of the body's response, it is advantageous if, subjected to tensile load, the implant of the invention expands in a main direction of expansion at Fₘₐₓ=16 N/cm by 30 to 40% and in a main direction of expansion at Fₘₐₓ=32 N/cm by 15 to 25%.

In that the expansion behavior is different in at least one main direction of expansion, it may be achieved, for example, that the implant has an adapted reduced expansion where it is oriented in immediate proximity to a bone.

At least two angularly offset main directions of expansion are hereby defined on the implant. The expansion behavior in the main directions of expansion is determined by thread spacing and/or thread thickness and/or thread material and/or mesh width. It may be appropriate to have the two main directions of expansion oriented normal to each other or to provide for further main directions of expansion.

In an advantageous embodiment of the invention, at least one of the main directions of expansion of the implant proposed is marked by a distinguishable thread and/or is color-marked. Commercially available meshes exhibit different properties, in particular different mechanical properties, in the various directions of expansion. These however are not marked as such so that the surgeon cannot recognize them. Although he may adapt the size of a mesh to the patient's defect and implant it into the body trunk, he cannot determine the orientation of the mesh so that the latter be mechanically adapted, in the best possible way, to the dynamometric conditions occurring at the repaired site. In providing such a marking, the surgeon is not only given the possibility to selectively choose the piece to be inserted with regard to the various expansion behaviors, which enables him to adapt the implant in the best possible way to the elastic properties of the surrounding body tissue, or with regard to an elastic behavior to be achieved. He may also selectively align implants displaying symmetry or even multiple symmetry (meaning more specifically such having round, square shapes and so on).

Moreover, such a marking is advantageous if an implant to be inserted passes through several hands during operation, greater transparency and, as a result thereof, controllability being achieved for all the persons involved in the operation by means of the marking of the invention.

The implant of the invention is further given advantageous suitability if it is configured as a planar structure. Thanks to the planar structure, the surgeon is particularly given the possibility to provide for potential connection sites for sutures on a plurality of locations and occurring forces may be diverted over the planar surface. In addition, body parts that are located away from the actual defect may be reached with the implant of the invention for the purpose of securing it, without too much space being needed therefore between the tissue structures of the body.

In an advantageous embodiment, the planar implant of the invention is characterized by a planar structure of a net-like configuration. In addition to the advantages described with respect to the purely planar structure, this type of embodiment also has the advantage of easy body tissue ingrowth into the interspaces of the net-like implant and of best possible integration of the implant with the body.

An implant in accordance with the invention also yields outstanding results when configured as a body. In some cases, a gap remains between the body structures adjacent to the repaired site even after insertion of a mesh. In hernia surgery for example, this gap often forms between the two rectus muscles. In this case, the dimensions of the gap are at least one centimeter in width, length and depth respectively. In such cases, the two rectus muscles must additionally be connected. Force taking net-like structures with wide tension sutures are used for the purpose. These are not only very inconvenient for the patient, they also are at high risk of mechanical failure on account of their length. Failure of the tension sutures imperatively calls for another surgical operation.

The body-shaped implant is particularly suited for supporting the neighboring tissue structures, meaning those located in the region of the repaired body orifice. Ideally, the shape of the body-shaped implant of the invention may be adapted in the best possible way to the dimensions of the defect, with the body-shaped implants more specifically being used in the form of cords, bars, cubes or spheres of different sizes. Gaping suture wounds may thus largely be avoided.

In a preferred variant of the body-shaped embodiment in accordance with the invention, the diameter or edge length extensions of the implant are intended to be in excess of 10 mm. For repairs in surgical operations intending to use the implant of the invention, smaller implant sizes are often unmanageable and moreover not appropriate.

It is particularly advantageous if the implant of the invention comprises protruding connection elements. The surgeon may use these connection elements to particularly easily establish connections with the body tissue surrounding the wound or with the body tissue intended to serve as an attachment point without the surgeon needing further aid in the form of means enabling him to connect the implant to the anchor points provided for. In that the connection elements are protruding, the shape of the implant may further be adapted to the dimensions of the wound to be closed, totally independent of the connection points chosen. Concurrently, the anchor points may be chosen independent of the size and location of the actual defect.

At least on its top and/or bottom side the implant may for example be comprised of laterally projecting connection segments. These connection segments may be integrally formed with the base body of the implant and at least partially overlap the adjoining tissue.

The implant may be dimensionally adapted to the given requirements by implementing it so as to be tailorable.

In a particularly advantageous embodiment of the implant of the invention, this is characterized by the fact that it comprises a body and a planar structure. In this variant, the body mainly performs the function of filling and supporting in the best possible way the gaping body orifice, whereas the planar part selectively promotes the ability to connect to, and integrate with, the body tissue.

An alloplastic implant of the type mentioned herein above may additionally be characterized in that it comprises, in addition to, or instead of, PVDF, a material selected from the group consisting of the following materials and/or the derivatives thereof: polyester-based polymer, polyglycolic acid, poly-tetra-fluor-ethylene, polyvinyl, polyamide, polypropylene, polyethylene, elastane, polyurethane, polyvinyl alcohol, polylactide, polyglycolide, polydioxanone, alginate, casein, protein, lactide/glycolide copolymers and other copolymers of the materials indicated.

Advantageous embodiments and an advantageous application of the invention will be described herein after with reference to the drawings.

In the drawings:

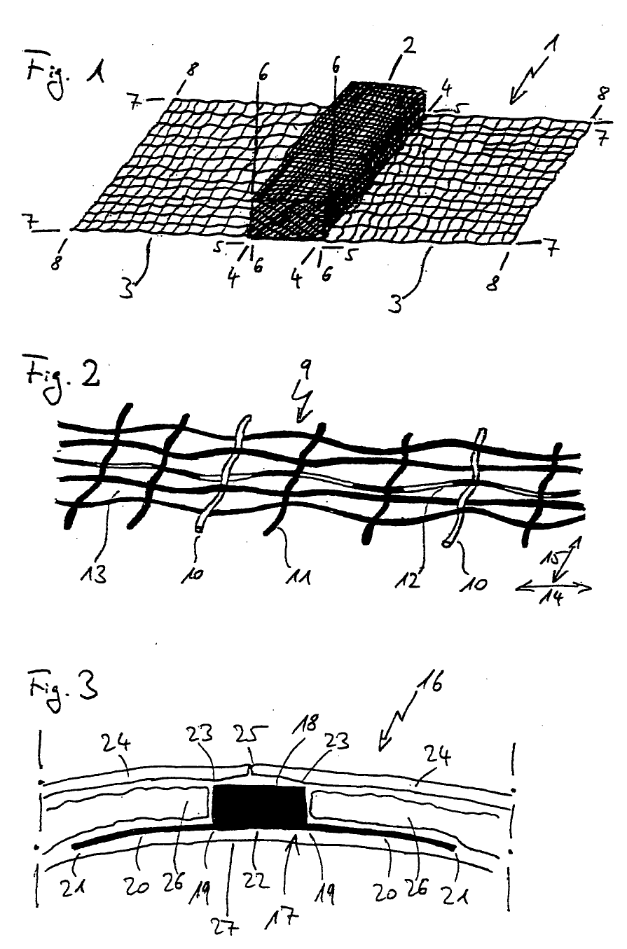

FIG. 1 shows a three-dimensional view of an exemplary implant,

FIG. 2 shows the three dimensional view of a small detail of a planar implant piece and

FIG. 3 shows a schematic diagram of a repaired abdominal wall using a proposed implant in a sectional view.

The implant 1 in FIG. 1 consists of a body-like, cord-shaped element 2 as well as of two laterally projecting planar implant pieces 3 that are attached at two edges 4 of the body-like piece. Both the planar elements 3 and the body-like part 2 of the implant 1 should consist, in the present example, of monofilament PVDF threads, e.g., even of identical material. The planar elements 3 are hereby configured as a net structure and are intended to serve as protruding connection elements for connecting the implant 1 to selected sites in the body by means of sutures or similar. In the exemplary embodiment shown, the length of every edge 4, 5 and 6 of the body 2 is in excess of one centimeter; the length of the edges 7 and 8 of the planar connection elements 3 is respectively of several centimeters in the example. In this embodiment, the body-like part 2 mainly performs the function of filling and supporting the gap in the human trunk.

FIG. 2 shows an exemplary embodiment of a planar net-shaped implant element in a detail view. The detail 9 that can be seen there may for example be any almost rectangular cutout of the planar connection elements 3 of the previous FIG. 1. The net structure formed by the threads 10, 11 and 12 and the resulting interspaces 13 is clearly apparent here. The respective one of the threads 12 and 10 is color-marked for identification of the two main directions of expansion 14 and 15 respectively. In the present example, the direction 15 is intended to be the stronger, meaning the less flexible, main direction of expansion, whereas the direction 14 is to correspond to the weaker main direction of expansion. The surgeon will recognize them by the marking of the threads: in the marking shown herein, the thread 10 has a completely different color and corresponds to the stronger main direction of expansion 15, whereas the thread 12, which is striped in the other color, corresponds to the weaker main direction of expansion 14. The rest of the network consists of the threads 11, which are all dyed the same color. In order to make the two threads marking the main direction of expansion 10 and 12 stand out, the other color used may for example be a signal color. The two marker threads 10 and 12 should be provided at regular intervals in the network 9. The marker threads 10 and 12 should be so closely parallelly spaced apart that, in the normal case, the chosen cutout always shows the two threads 10, 12 even if the implant piece is relatively small.

FIG. 3 shows an abdominal wall closure as an example of how to repair a damaged body trunk 16 using an implant 17 in accordance with the invention. In the example, the implant 17 again consists of a body-like element 18 and of two laterally protruding planar connection elements 20 that are fastened at the edges 19. More specifically for tangentially securing the implant, sutures can be placed at the sites 21 for securing the implant to the underlying rear sheath of the rectus muscle 27. An additional suture may be placed on site 22, more specifically in order to secure the implant in the radial direction. Then, the cord-shaped part 18 of the implant 7 can be sutured at the sites 23 to the front abdominal muscles 26 and thereafter the opening of the skin 24 at site 25 be reliably closed. It can clearly be seen from this exemplary embodiment how the body-like part 18 of the implant 17 is capable of supporting the neighboring tissue 26 and of filling out the gap there. This, together with the additional fastening of the muscles 26 to the sites 23, permits to successfully prevent the skin site 25 to be closed from gaping. It is further obvious therefrom that it is very improbable that the suture 25 be still subjected to mechanical load. The implant 17 is allowed to grow together with the body tissue thanks to the porous base structure along the entire surface of both the body-like element 18 and the planar connection elements 20. This permits well-tolerated complete integration of the implant 17 with the body trunk.

It is to be understood that the embodiments of the textile implant material shown may also comprise, at least in parts, a resorbable material or a biocompatible material or be coated at least partially without this limiting what has been described herein above.

1. A textile implant material characterized in that it comprises monofilament threads of polyvinylidene fluoride (PVDF) or of a PVDF derivative.

2. The textile implant material according to claim 1, characterized in that it is mainly formed from monofilament threads.

3. The textile implant material according to claim 1, characterized in that it has a porous structure.

4. The textile implant material according to claim 1, characterized in that it has a pore size of from 1 to 5 mm, preferably from 1 to 3 mm.

5. The textile implant material according to claim 1, characterized in that the implant comprises a woven fabric, a knit fabric, a knitting, a layered fabric, a braided fabric or a nonwoven fabric.

6. The textile implant material according to claim 1, characterized in that it has a flexible structure.

7. The textile implant material according to claim 1, characterized in that at least part of the implant comprises a resorbable material.

8. The textile implant material according to claim 1, characterized in that at least part of the implant comprises a biocompatible material.

9. The textile implant material according to claim 1, characterized in that at least part of the implant comprises a coating.

10. The textile implant material according to claim 1, characterized in that, subjected to tensile load, the implant expands in a main direction of expansion at Fₘₐₓ=16 N/cm by 30 to 40% and in a main direction of expansion at Fₘₐₓ=32 N/cm by 15 to 25%.

11. The textile implant material according to claim 1, characterized in that at least one of the main directions of expansion is marked by a distinguishable thread and/or is color-marked.

12-13. (canceled).

14. The textile implant material according to claim 1, characterized in that it is configured as a body.

15. The textile implant material according to claim 14, characterized in that the diameter or edge length extensions of the body are in excess of 10 mm.

16. The textile implant material according to claim 1, characterized in that it comprises protruding connection elements.

17 (canceled).
